(19) United States
(12) Patent Application Publication
(10) Pub. No.: US 2004/0116405 A1
(43) Pub. Date: Jun. 17, 2004

(54) Use of bicyclic esters or amides for the treatment of diseases responsive to enhanced potassium channel activity

(76) Inventors: Brian Cox, Horsham (GB); Jennifer Elizabeth Cryan, Stevenage (GB); Timothy James Dale, Stevenage (GB); Graeme Michael Robertson, Stevenage (GB); Derek John Trezise, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/467,254

(22) PCT Filed: Feb. 4, 2002
(86) PCT No.: PCT/GB02/00496

(30) Foreign Application Priority Data
Feb. 6, 2001 (GB) 0102927.1
Feb. 6, 2001 (GB) 0102928.9

(51) Int. Cl. A61K 31/5377; A61K 31/4745; C07D 413/02

(52) U.S. Cl. 514/210.21; 514/232.8; 514/291; 544/126; 546/79

(57) ABSTRACT

A compound of formula (I): or a salt, solvate or physiologically functional derivative thereof for use as a IK and/or SK channel opener. Methods of screening SK ion channels, particularly SK1 and medicaments for the treatment of a urinogenital, respiratory, cardiovascular, neuronal disorder, of sleeping disorders, sickle cell anemia, pain, inflammation and bowel disease (IBD) are also disclosed.

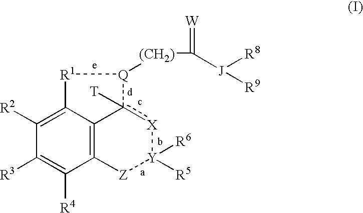

### FIELD F THE INVENTION

The invention relates to openers of calcium activated potassium channels and their use in the treatment of disorders responsive to enhanced calcium activated potassium channel activity. The invention also relates to screening methods for the identification of modulators of the channels described supra, methods of medical treatment and medicaments comprising the same.

### BACKGROUND OF THE INVENTION

Calcium activated potassium (K⁺) channels play an important role in controlling the excitability and function of many cell types including neurones, epithelial cells, T-lymphocytes and skeletal muscle. Calcium activated K⁺ channels are activated by submicromolar increases in intracellular calcium and this activation is modulated by calmodulin. In excitable cells, activation of SK channels generates a hyperpolarizing K⁺ current which contributes to the after-hyperpolarization (AHP) that follows an action potential. This AHP modulates cell firing frequency and spike frequency adaptation thereby influencing neuronal excitability. SK channels have been implicated in diverse physiological and pathophysiological functions such as learning and memory, synaptic enhancement and long-term potentiation.

Calcium activated potassium channels can be divided into three main classes.

Large conductance (BK), intermediate conductance (IK) and small conductance (SK) calcium activated K⁺ channels. SK channels belong to the class of ligand-gated ion channels and to date three subtypes (SK-1, SK-2, SK-3) have been identified. The cloning of IK and SK-1, SK-2 and SK-3 channels has been described in WO 98/11139. SK channels are have a small single channel conductance for potassium of less than 25 pS and are voltage-independent. The bee venom peptide toxin apamin specifically blocks SK-2 and SK-3 channels but not SK-1 channels. IK channels are voltage insensitive and can be blocked by charybdotoxin and clotrimazole. See WO98/11139 for the cloning, expression and sequence characteristics of IK and SK ion channels. This document is specifically incorporated in its entirety and the reader is specifically referred to it.

### SUMMARY F INVENTION

The present invention is based on the finding that certain compounds act as openers of SK and IK channels and that these compounds may therefore be useful in the treatment of disorders responsive to enhanced IK and/or SK channel activity.

Accordingly the present invention provides:

a compound of formula (I):

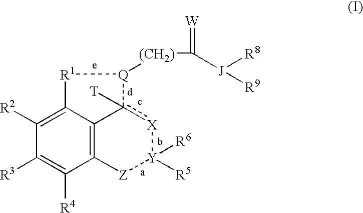

wherein

R², R³ and R⁴, which are the same or different are each hydrogen, hydroxy, halogen, cyano, C₁₋₆ alkyl, C₁₋₆ alkoxy or C₁₋₆ haloalkyl;

R⁸ and R⁹, which are the same or different are each hydrogen, C₃₋₅ cycloalkyl, C₁₋₆ alkoxy, phenyl, benzyl or C₁₋₆ alkyl which is unsubstituted or substituted by hydroxy; or R⁸ and R⁹ together with J form a morpholino group, a pyrrolidine group or an azetidine group;

n is 0 or 1;

W is 0 or S; and

J is 0 or N; and

(a) _{——}e_{——} is a single bond, _{——}d_{——} is a double bond, and _{——}a_{——}, _{——}b_{——} and _{——}c_{——} are absent;

R⁵, R⁶, T and Y are absent;

R¹ is CH₂ or O;

X is hydrogen or C₁₋₆ alkyl; Z is as defined above for R², R³ and R; and

Q is carbon, or

(b) _{——}e_{——} is absent;

_{——}d_{——} is a single bond or a double bond, _{——}c_{——} is a bond or is absent, provided that when _{——}d_{——} is a double bond, _{——}c_{——} and T are absent; and when _{——}d_{——} is a single bond one of T and _{——}c_{——} is absent;

_{——}b_{——} is a single bond and _{——} a_{——} is a single bond or is absent, or

_{——}a_{——} Y(R⁵)(R⁶)_{——}b_{——}represents a single bond;

Q is CH when _{——}d_{——} is a double bond and CH₂ when _{——}d_{——} is a single bond;

T is hydrogen or hydroxy;

X is CR⁷ or CHR⁷, wherein R⁷ is hydrogen, hydroxy, halogen, nitroxy, C₁₋₆ acyloxy or C₁₋₄ alkoxy;

Y is C or CH;

Z is (i) CH₂, oxygen or sulphur when _{——}a_{——} is a bond; or (ii) is hydrogen, halogen or C₁₋₆alkyl when _{——}a_{——} is absent; or (iii) the moiety Z_{——}a_{——} represents a bond when R⁵ and R⁶ together with Y form a carbonyl group;

R¹ is as defined above for R², R³ and R⁴;

R⁵ is hydrogen or C₁₋₆ alkyl;

R⁶ is hydrogen, C₁₋₆ alkyl, hydroxy or halogen, or R⁵ and R⁶ together with Y form a carbonyl group; or

(c) _{——}e_{——} is absent; _{——}d_{——} is a single bond or a double bond, provided that when _{——}d_{——} is a double bond, T is absent;

_{——}c_{——} is absent; and

_{——}a_{——} Y(R⁵)(R⁶)_{——}b_{——} is absent;

Q and T are as defined in (b) above;

X is hydrogen or C₁₋₆ alkyl;

Z is hydrogen, halogen or C₁₋₆ alkyl; and

R¹ is as defined above for R², R³ and R⁴;

or a salt, solvate or physiologically functional derivative thereof for use as an IK and/or a SK channel opener; thus a compound of the invention may be used in a method of activating IK and/or SK channels;

use of a compound of formula (I) in the manufacture of a medicament for the treatment of a disorder responsive to enhanced IK and/or SK channel activity;

method for treating a patient afflicted with a disorder responsive to enhanced IK and/or SK channel activity, the said method comprising administering to a patient in need of treatment a therapeutically effective amount of a compound of formula (I).

Preferably the disorder is selected from urinogenital disorders such as bladder hyperexcitability, MED and urinary incontinence, respiritory disorders such as asthma, chronic obstructive pulmonary disease (COPD) and cystic fibrosis, cardiovascular disorders such as hypertension, angina pectoris, ischaemic heart disease and cerebral ischaemia, neuronal hyperexcitability disorders such as irritable bowel syndrome (IBS), bipolar disorder and psychosis, sickle cell anaemia, sleeping disorders and hyperalkaemia. Furthermore the disorder can be selected from a disorder of the central nervous system such as epilepsy, inflammation, pain and inflammatory bowel disease (IBD).

Bicyclic amide derivatives are disclosed in WO94/26693 as potent central muscle relaxants. WO95/30644 discloses novel carbocyclic amides together with their salts, solvates and physiologically active derivatives as central muscle relaxants and in the treatment of anxiety, inflammation, arthritis and pain. WO95/30645 discloses a group of substituted carbocyclic amides in particular in the treatment of inflammatory conditions. These references are in particular, incorporated herein by reference. The reader is specifically referred to each reference.

In another aspect of the present invention there is provided a method for the identification of a candidate agent useful in, inter alia, the treatment of a disease or disorder selected from the group consisting of; pain, urinogenital disorders such as bladder hyperexcitability, MED and urinary incontinence, respiratory disorders such as asthma, chronic obstructive pulmonary disease (COPD) and cystic fibrosis, cardiovascular disorders such as hypertension, angina pectoris, ischaemic heart disease and cerebral ischaemia, neuronal hyperexcitability disorders such as irritable bowel syndrome, bipolar disorder and psychosis, sickle cell anaemia, sleeping disorders and hyperalkaemia which method comprises the steps of;

(a) providing a candidate agent;

(b) contacting a SK-1 channel with said candidate agent;

(c) selecting a candidate agent which modulates opening of said SK-1 channel;

(d) synthesising and/or purifying said agent of step (c).

A further aspect of the invention is the above method for the identification of a candidate agent useful in the treatment of disorder can be selected from a disorder of the central nervous system such as epilepsy, inflammation, pain and inflammatory bowel disease (IBD).

In another aspect of the present invention, we provide a method for the treatment of a disease or disorder selected from the group consisting of; pain, urinogenital disorders such as bladder hyperexcitability, MED and urinary incontinence, respiratory disorders such as asthma, chronic obstructive pulmonary disease (COPD) and cystic fibrosis, cardiovascular disorders such as hypertension, angina pectoris, ischaemic heart disease and cerebral ischaemia, neuronal hyperexcitability disorders such as irritable bowel syndrome, bipolar disorder and psychosis, sickle cell anaemia, sleeping disorders and hyperalkaemia which method comprises the steps of;

(a) providing a candidate agent;

(b) contacting a SK-l channel with said candidate agent;

(c) selecting a candidate agent which modulates (i.e. promotes) opening of said SK-1 channel;

(d) synthesising and/or purifying said agent of step (c).

(e) administering said agent of step (c) or (d) or a modified version thereof to a mammalian patient, particularly a human patient, in clinical need thereof.

A further aspect of the invention is the above method for the identification of a candidate agent useful in the treatment of disorder can be selected from a disorder of the central nervous system such as epilepsy, inflammation, pain and inflammatory bowel disease (IBD).

### BRIEF DESCRIPTION OF THE DRAWINGS

FIG. 1 illustrates the activation of hIK channels expressed in CHO—K1 cells by a compound of formula (V):

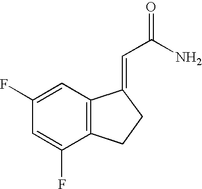

FIG. 1(a) shows whole cell currents;

FIG. 1(b) shows the timecourse of activation;

FIG. 1(c) shows concentration response curves in 4 different cells; and

FIG. 1(d) shows inhibition by the IK blocker, charybdotoxin (100 nM).

### DETAILED DESCRIPTION OF THE INVENTION

The invention provides compounds of formula (I) for use as calcium activated potassium channel openers:

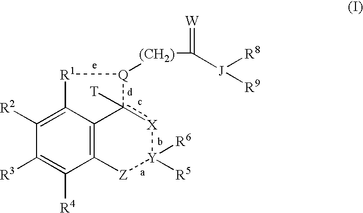

wherein

R², R³ and R⁴, which are the same or different are each hydrogen, hydroxy, halogen, cyano, C₁₋₆ alkyl, C₁₋₆ alkoxy or C₁₋₆ haloalkyl;

R⁸ and R⁹, which are the same or different are each hydrogen, C₃-₅ cycloalkyl, C₁₋₆ alkoxy, phenyl, benzyl or C₁₋₆ alkyl which is unsubstituted or substituted by hydroxy; or R⁸ and R⁹ together with J form a morpholino group, a pyrrolidine group or an azetidine group;

n is 0 or 1;

W is O or S; and

J is O or N; and

(a) _{——}e_{——} is a single bond, _{——}d_{——} is a double bond, and _{——}a_{——}, _{——}b_{——} and _{——}c_{——} are absent;

R⁵, R⁶, T and Y are absent;

R¹ is CH₂ or O;

X is hydrogen or C₁₋₆ alkyl; Z is as defined above for R², R³ and R⁴ ; and Q is carbon, or

(b) _{——}e_{——} is absent;

_{——}d_{——} is a single bond or a double bond, _{——}c_{——} is a bond or is absent, provided that when _{——}d_{——} is a double bond, _{——} c_{——} and T are absent; and

when _{——}d_{——} is a single bond one of T and _{——}c_{——} is absent;

_{——}b_{——} is a single bond and _{——}a_{——} is a single bond or is absent, or

_{——}a_{——} Y(R⁵)(R⁶)_{——}b_{——} represents a single bond;

Q is CH when _{——}d_{——} is a double bond and CH₂ when _{——}d_{——} is a single bond;

T is hydrogen or hydroxy;

X is CR⁷ or CHR⁷, wherein R⁷ is hydrogen, hydroxy, halogen, nitroxy, C₁₋₆ acyloxy or C₁₋₄ alkoxy;

Y is C or CH;

Z is (i) CH₂, oxygen or sulphur when _{——}a_{——} is a bond; or (ii) is hydrogen, halogen or C₁₋₆ alkyl when _{——}a_{——} is absent; or (iii) the moiety Z_{——}a_{——} represents a bond when R⁵ and R⁶ together with Y form a carbonyl group;

R¹ is as defined above for R², R³ and R⁴;

R⁵ is hydrogen or C₁₋₆ alkyl;

R⁶ is hydrogen, C₁₋₆ alkyl, hydroxy or halogen, or R⁵ and R⁶ together with Y form a carbonyl group; or

(c) _{——}e_{——} is absent; _{——}d_{——} is a single bond or a double bond, provided that when _{——}d_{——} is a double bond, T is absent;

_{——}c_{——} is absent; and

_{——}a_{——} Y(R⁵)(R⁶)_{——}b_{——} is absent;

Q and T are as defined in (b) above;

X is hydrogen or C₁₋₆ alkyl;

Z is hydrogen, halogen or C₁₋₆ alkyl; and

R¹ is as defined above for R², R³ and R⁴;

or a salt, solvate or physiologically functional derivative thereof.

Typically J is nitrogen and W is oxygen.

In a preferred embodiment of formula (I) _{——}a_{——} and _{——}b_{——} are present and _{——}c_{——} is absent.

In another preferred embodiment one of R⁸ and R⁹ is cyclopropyl, cyclobutyl or cyclopentyl, preferably cyclopropyl.

In another preferred embodiment the compounds are the compounds of formula (II):

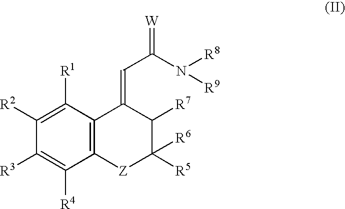

wherein

R¹, R², R³ and R⁴ are each independently hydrogen, halogen, cyano, C₁₋₆ alkyl, C₁₋₆ alkoxy, or C₁₋₆ haloalkyl;

R⁵ is hydrogen or C₁₋₆ alkyl;

R⁶ is hydrogen, C₁₋₆ alkyl, hydroxy, or halogen;

or R⁵ and R⁶ together with the carbon atom to which they are attached form a carbonyl group;

R⁷ is hydrogen, hydroxy, C₁-C₄ alkoxy, halogen or an acyloxy group;

R⁸ and R⁹ are each independently hydrogen, C₃₋₅ cycloalkyl, C₁₋₆ alkoxy, benzyl, phenyl, or C₁₋₆ alkyl which is unsubstituted or substituted by hydroxy;

or R⁸ and R⁹ together with the nitrogen to which they are bonded form a morpholino group, a pyrrolidine group or an azetidine group;

Z is a bond, CH₂, —O— or —S— and is always a bond when R⁵ and R⁶ together with the carbon to which they are attached form a carbonyl group; and

W is O or S;

or a salt, solvate or physiologically functional derivative thereof.

Prefered compounds of formula (II) are compounds of formula (IIa);

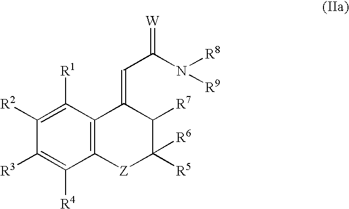

wherein

R¹, R², R³ and R⁴ are each selected from hydrogen, and fluoro and at least one and not more than two is fluoro;

R⁵ is selected from hydrogen or C₁₋₄ alkyl and R⁶ is hydrogen, C₁₋₄ alkyl, hydroxy, or R⁵ and R⁶ together with the carbon atom to which they are attached form a carbonyl group;

R⁷ is hydrogen, hydroxy;

R⁸ and R⁹ are each independently hydrogen, C₁₋₄ alkyl and C₃ or C₄ cycloalkyl, or together with the nitrogen to which they are bonded form a morpholino group;

or a salt, solvate or physiologically functional derivative thereof.

In another preferred embodiment the compounds are the compound of formula (III):

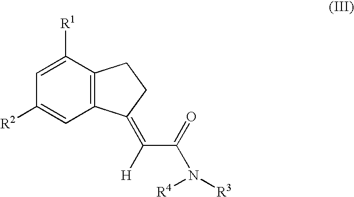

wherein

R¹ and R² are independently selected from chloro, fluoro, bromo, C₁₋₆ alkyl, C₁₋₆ alkoxy or C₁₋₆haloalkyl provided that both R¹ and R² are not fluoro; and

R³ and R⁴ are independently selected from hydrogen and C₁₋₆ alkyl;

or a salt, solvate or physiologically functional derivative thereof; or

In another preferred embodiment the compounds are the compounds of formula (IV):

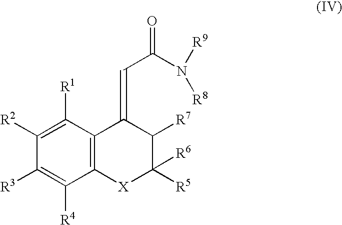

wherein

R¹, R², R³ and R⁴ are each selected from hydrogen and fluoro and at least one and not more than two is fluoro;

R⁵ is selected from hydrogen and C₁₋₄ alkyl and R⁶ is selected from hydrogen, C₁₋₄ alkyl and hydroxy or R⁵ and R⁶ together with the ring carbon form a carbonyl group;

R⁷ is selected from hydrogen and hydroxy;

R⁸ and R⁹ are each selected from hydrogen, C₁₋₄ alkyl and cyclo(C₃ or C₄) alkyl or together with the nitrogen form a morpholino group; and

X is selected from a bond, methylene and —O— and is always a bond or —O— when any of R⁵, R⁶ and R⁷ is other than hydrogen and is always a bond when R⁵ and R⁶ together with the ring carbon form a carbonyl group;

or a salt, solvate or physiologically functional derivative thereof.

Other preferred compounds are:

(E)-2-(4-Chloro-1-indanylidene)acetamide,

(E)-2-(4-Methyl-1-indanylidene)acetamide,

(E)-1-(2-(6-Fluoro-1-indanylidene)acetyl)pyrolidine,

(E)-2-(6-Fluoro-1-indanylidene)-N-phenylacetamide,

(E)-1-(2-(6-Fluoro-1-indanylidene)acetyl)azetidine,

(E)-2-(6-Fluoro-1-indanylidene)-N-methoxy-N-methylacetamide,

(Z)-2-(6-Fluoro-2-nitroxy-1-indanylidene)acetamide,

(E)-2-(6-Fluoro- -indanylidene)-N-(2-hydroxyethyl)acetamide,

(Z)-2-(6-Fluoro-2-methoxy-1-indanylidene)acetamide,

(Z)-2-(2,3-dibromo-6-fluoro-1-indanylidene)acetamide,

(E)-2-(6-Fluoro-1-indanylidene)thioacetamide,

(E)-N-Cyclopentyl-2-(6-fluoro-1-indanylidene)acetamide,

(Z)-2-(2-Acetoxy-6-fluoro-1-indanylidene)acetamide,

(Z)-2-(2-Bromo-6-fluoro-1-indanylidene)acetamide,

(E)-2-(4,6-Difluoro-1-indanylidene)-N-(2-hydroxyethyl)acetamide,

(E)-N-Cyclopropyl-2-(7-methyl-1-indanylidene)acetamide,

(E)-2-(6-Cyano-1-indanylidene)acetamide,

(Z)-2-(2-Bromo-4,6-difluoro-1-indanylidene)acetamide,

(E)-2-(6-Bromo-1-indanylidene)-N-cyclopropylacetamide,

(E)-2-(6-Bromo-1-indanylidene)N-methylacetamide,

(E)-2-(6-Methoxy-1-indanylidene)acetamide,

(E)-2-(7-Chloro-1,2,3,4-tetrahydro-1-naphthylidene)-N-cyclopropylacetamide,

(E)-2-(5-Bromo-1,2,3,4-tetrahydro-1-naphthylidene)-N-cyclopropylacetamide,

(E)-N-Cyclopropyl-2(1,2,3,4-tetrahydro-7-methoxy-1-naphthylidene)acetamide,

(E)-N-Cyclopentyl-2-(7-fluoro-1,2,3,4-tetrahydro-1-naphthylidene)acetamide,

(E)-N-Cyclopropyl-2-(1,2,3,4-tetrahydro-6-methoxy-1-naphthylidene)acetamide,

(E)-N-Benzyl-2-(7-fluoro-1,2,3,4-tetrahydro-1-naphthylidene)acetamide,

(E)-N-Cyclopropyl-2-(5-fluoro-1,2,3,4-tetrahydro-1-naphthylidene)propionamide,

(E)-2-(7-Fluoro-1,2,3,4-tetrahydro-1-naphthylidene)-N-(2-hydroxyethyl)acetamide,

(E)-2-(6-Fluoro-1,2,3,4-tetrahydro-1-naphthylidene)-N-methyl-N-methoxyacetamide,

(Z)-N-Cyclopropyl-2-(7-fluoro-1,2,3,4-tetrahydro-2,2-dimethyl-1-naphthylidene)acetamide,

(E)-N-Cyclopropyl-2-(7-fluoro-1,2,3,4-tetrahydro-2,2-dimethyl-1-naphthylidene)acetamide,

(E)-2-(7-Fluoro-1,2,3,4-tetrahydro-1-naphthylidene)acetamide,

(E)-1-(2-(7-Fluoro-1,2,3,4-tetrahydro-1-naphthylidene)acetyl)azetidine,

(E)-1-(2-(7-Fluoro-1,2,3,4-tetrahydro-1-naphthylidene)acetyl)pyrrolidine,

(E)-N-Cyclopropyl-2-(1,2,3,4-tetrahydro-7-methyl-1-naphthylidene)acetamide,

(E)-N-Cyclopropyl-2-(1,2,3,4-tetrahydro-5-methoxy-l-naphthylidene)acetamide,

(Z)-N-Cyclopropyl-2-(1,2,3,4-tetrahydro-1-naphthylidene)acetamide,

(E)-2-(6-Chloro-1,2,3,4-tetrahydro-1-naphthylidene)-N-cyclopropylacetamide,

(Z)-2-(7-Chloro-1,2,3,4-tetrahydro-1-naphthylidene)-N-cylcopropylacetamide,

(Z)-N-Cyclopropyl-2-(1,2,3,4-tetrahydro-7-methoxy-1-naphthylidene)acetamide,

(Z)-N-Cyclopentyl-2-(7-fluoro-1,2,3,4-tetrahydro-1-naphthylidene)acetarnide,

(Z)-2-(7-Fluoro-1,2,3,4-tetrahydro-1-naphthylidene)-N-phenylacetamide,

(E)-2-(6-Chloro-3,4-dihydro-2H-1-benzopyran-4-ylidene)-N-cyclopropylacetamide,

(E)-2-(6-Chloro-3,4-dihydro-2H-1-benzopyran4-ylidene)-N-methylacetamide,

(E)-2-(6-Chloro-3,4-dihydro-2H-1-benzopyran4-ylidene)acetamide,

(E)-N-Cyclopropyl-2-(3,4-dihydro-2H-1-benzothiopyran4-ylidene)acetamide,

(E)-N-2-(3,4-dihydro-2H-1-benzothiopyran4-ylidene)-N-methylacetamide,

(E)2-(3,4-dihydro-2H-1-benzothiopyran-4-ylidene)acetamide and

(E)-N-Cyclopropylmethyl-2-(7-fluoro-1,2,3,4-tetrahydro-1-naphthalidene)acetamide,

or a salt or solvate or physiologically functional derivative thereof.

A preferred group of compounds are compounds of formula (III) is that of the formula (IIIa):

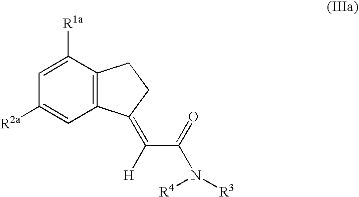

wherein

R^{1a} is chloro, R^{2a} is chloro, fluoro, bromo or methyl; and

R³ and R⁴ are independently selected from hydrogen, methyl and ethyl.

Preferred compounds within formula (1) include:

(E)-2-(4-chloro-6-fluoro-1-indanylidene)-N-methylacetamide;

(E)-2-(4-chloro-6-fluoro-1-indanylidene)acetamide;

(E)-2-(4,6-dichloro-1-indanylidene)acetamide;

(E)-2-(6-fluoro4-methyl-1-indanylidene)acetamide;

(E)-2-(6-fluoro4-methyl-1-indanylidene)-N-methylacetamide;

(E)-2-(6-chloro4-fluoro-1-indanylidene)acetamide;

(E)-2-(4-bromo-6-fluoro-1-indanylidene)acetamide;

(E)-2-(4-chloro-6-methyl-1-indanylidene)acetarnide.

Separate groups of compounds within formula (IV) include those wherein

(i) one of R¹, R², R³ and R⁴ is fluoro;

(ii) two of R¹, R², R³ and R⁴ are fluoro;

(iii) R¹ is fluoro;

(iv) R² is fluoro;

(v) R³ is fluoro;

(vi) R⁴ is fluoro;

(vii) R⁵ is hydrogen;

(viii) R⁵ is Coo alkyl, preferably alkyl having 1, 2 or 3 carbon atoms and more preferably methyl or ethyl;

(ix) R⁶ is hydrogen;

(x) R⁶ is C₁₋₄ alkyl, preferably alkyl having 1, 2 or 3 carbon atoms and more preferably methyl or ethyl;

(xi) R⁶ is hydroxy;

(xii) R⁵ and R⁶ together with the ring carbon form a carbonyl group;

(xiii) R⁷ is hydrogen;

(xiv) R⁷ is hydroxy;

(xv) R⁸ is hydrogen;

(xvi) Rs is C₁₋₄ alkyl, preferably alkyl having 1, 2 or 3 carbon atoms and more preferably methyl, ethyl or isopropyl;

(xvii) R⁸ is cyclo(C₃ or C₄)alkyl, preferably cyclopropyl;

(xviii) R⁹ is hydrogen;

(ixx) R⁹ is C₁₋₄ alkyl, preferably alkyl having 1, 2 or 3 carbon atoms and more preferably methyl, ethyl or isopropyl;

(xx) R⁹ is cyclo(C₃ or C₄)alkyl, cyclopropyl;

(xxi) R⁸ and R⁹ together with the nitrogen form a morpholino group;

(xxii) X is a bond;

(xxiii) X is methylene;

(xxiv) X is —O—.

Preferred as a class of compounds wherein the >C═O group and the benzene ring are on opposite sides of the exo double bond.

Individual preferred compounds within formula (II) include:

(E)-2-(6-fluoro-3-methyl-1-indanylidene)acetamide;

(E)-N-cyclopropyl-2-(6-fluoro-3-methyl-1-indanylidene)acetamide;

(E)-2-(6-fluoro-3,3-dimethyl-1-indanylidene)-N-methylacetamide;

(E)-N-cyclopropyl-2-(6-fluoro-3-ethyl-1-indanylidene)acetamide;

(E)-N-cyclopropyl-2-5,6-difluoro-1-indanylidene)acetamide;

(E)-2-(5,6-difluoro-1-indanylidene)-N-methylacetamide;

(E)-2-(5,6-difluoro-1-indanylidene)acetamide;

(E)2-(5,7-difluoro-1-indanylidene)acetamide;

(E)-N-cyclopropyl-2-(4,6-difluoro-1-indanylidene)acetamide;

(E)-2-(4,6-difluoro-1-indanylidene)-N-isopropylacetamide;

(E)-2-(4,6difluoro-1-indanylidene)-N,N-dimethylacetamide;

(Z)-2-(4,6-difluoro-2-hydroxy-1-indanylidene)acetamide;

5 (E)-2-(7-fluoro-1,2,3,4-tetrahydro-1-naphthylidene)acetamide;

(E)-N-cyclopropyl-2-(7-fluoro-1,2,3,4-tetrahydro-1-naphthylidene)acetamide;

(E)-N-cyclopropyl-2-(6-fluoro-3,4-dihydro-2H-1-benzopyran4-ylidene)acetamide;

(E)-2-(4,6-difluoro-1-indanylidene)acetamide;

(E)-2-(6-fluoro-1-indanylidene)acetamide;

(Z)-2-(6-fluoro-2-hydroxy-1-indanylidene)acetamide;

(E)-2-(6-fluoro-3,3dimethyl-1 -indanylidene)acetamide;

(E)-2-(6-fluoro-3-ethyl-1-indanylidene)-N,N-dimethylacetamide;

(E)-2-(6-fluoro-3-hydroxy-1-indanylidene)acetamide.

Particularly preferred is (E)-2-(4,6-difluoro-1-indanylidene)acetamide. As used herein the term:

(a) “C₁₋₆ alkyl” means an alkyl group having from 1 to 6 carbon atoms, for example a C₁₋₄ alkyl group having 1, 2, 3 or 4 carbon atoms. Such a group may be a linear, branched chain or cyclic alkyl group. Suitable alkyl groups preferably have 1 to 4 carbon atoms and are more preferably methyl, ethyl, propyl, prop-2-yl, butyl, but-2-yl, or 2-methylprop-2-yl. Alkyl groups are most preferably methyl or ethyl.

(b) “C₁₋₆ alkoxy” as a group or part of a group means a monvalent straight or branched chain radical having from 1 to 6 carbon atoms which are attached to the parent moiety through an oxygen atom. Such alkoxy groups preferably have 1 to 4 carbon atoms and are more preferably methoxy or ethoxy, most preferably methoxy.

(c) “C₁₋₆ haloalkyl” means a C₁₋₆ alkyl group as defined in (a) substituted by 1 to fluoro or chloro atoms preferably fluoro atoms, for example, 1, 2, 3, 4 or 5 fluoro atoms. Suitable groups include perfluoro groups such as trifluoromethyl.

(d) “C₃₋₅cycloalkyl” means a cycloalkyl group having 3, 4 or 5 carbon atoms, for instance cyclopropyl or cyclobutyl. Preferably it is cyclopropyl.

(e) “C₁₋₆ acyloxy” means a straight or branched chain acyl group of 1 to 6 carbon atoms which is attached to the parent moiety through an oxygen atom. Such acyloxy groups preferably have 1 to 4 carbon atoms and are preferably acetoxy.

(f) “Halogen” means fluorine, chlorine, bromine or iodine, preferably fluorine, chlorine or bromine.

(g) “salt” means a base salt as further defined herein below. In relation to compounds of formula (II), “salt” means a base salt formed when one of R⁸ and R⁹ is hydrogen.

(h) “solvate” means a combination, in definite proportions, of a compound of the present invention with its solvent.

(i) “physiologically functional derivatives” means any other physiologically acceptable derivative of a compound of the present invention, for example an ester, which upon administration to the recipient, such as a human, is capable of providing (directly or indirectly) the said compound or an active metabolite thereof.

It will be appreciated that compounds of the invention can exist in various geoisometric forms or mixtures of geoisomers, including the individual E and Z isomers of the compounds of the invention as well as mixtures of such isomers, in any proportions. Preferred compounds of formula (I) are those wherein the group adjacent to the exo double bond and the carbonyl group are on opposite sides of the exo double bond. The compounds of the invention may exist in forms wherein one or more carbon centre is/are chiral. The present invention includes within its scope each possible optimal isomer substantially free, i.e., associated with less than 5% of any other optimal isomer(s), as well as mixtures of one or more optical isomers in any proportion, including racemic mixtures thereof.

Pharmaceutically acceptable salts are within the scope of the invention and are particularly suitable for medical applications because of their greater aqueous solubility relative to the parent (i.e. basic) compounds. Such salts must clearly have a pharmaceutically acceptable anion or cation. Suitable pharmaceutically acceptable base salts include ammonium salts, alkali metal salts, such as sodium or potassium salts, and alkaline earth salts, such as magnesium and calcium salts. Salts having a non-pharmaceutically acceptable anion are also within the scope of the invention as useful intermediates for the preparation or purification of pharmaceutically acceptable salts in the process of manufacturing a medicament.

A compound of the invention typically acts as an IK and or an SK channel opener, preferably an SK-1 channel opener. An IK channel opener is a compound which activates IK channels. A SK channel opener is a compound which activates SK channels. An IK or SK channel is activated when it opens to allow potassium ions to flow through the channel. Preferably a compound of the invention opens IK and/or SK channels in a Ca²⁺ sensitive manner. A compound that opens IK and/or SK channels in a Ca²⁺ sensitive manner typically activates K⁺ channel activity of the channel only in the presence of Ca²⁺.

A compound of the invention may activate IK, SK-1, SK-2 and SK-3 channels or may selectively activate one or more channel subtype. For example a compound of the invention may selectively activate SK channels. A compound which selectively activates SK channels will activate SK channels but will not activate or will activate less strongly IK channels. Alternatively a compound of the invention may activate IK channels. A compound which selectively activates IK channels will activate IK channels but will not activate or will activate less strongly SK channels. A compound may selectively activate a subtype of SK channels. For example a compound which selectively activates SK-l channels will activate SK-1 channels but will not activate or will activate only to a lesser extent SK-2 and SK-3 channels.

The present invention provides a method of treating a patient afflicted with a disorder responsive to enhanced IK and/or SK channel activity, which method comprises administering a therapeutically effective amount of compound of formula (I) or a compound identified by the screening method of the present invention to a patient in need of treatment. A patient in need of treatment will typically be afflicted with a urinogenital disorder such as bladder hyperexcitability, MED or urinary incontinence, a respiritory disorder such as asthma, chronic obstructive pulmonary disease (COPD) or cystic fibrosis, a cardiovascular disorder such as hypertension, angina pectoris, ischaemic heart disease or cerebral ischaemia, a neuronal hyperexcitability disorder such as irritable bowel syndrome, bipolar disorder or psychosis, sickle cell anaemia, sleeping disorders and hyperalkaemia. A further aspect of the invention a method of treating a disorder of the central nervous system such as epilepsy, inflammation, pain and inflammatory bowel disease (IBD).

Administration of a compound of the invention to a patient afflicted with a disorder responsive to enhanced IK and/or SK channel activity will typically improve the condition of a patient afflicted with the disorder and/or alleviate the symptoms of the disorder.

The invention also provides the use of a compound of the invention in the manufacture of a medicament for the treatment of a disorder responsive to enhanced IK and/or SK channel activity.

Use of a SK-1 ion channel activator, in the manufacture of a medicament for the treatment of pain and/or irritable bowel syndrome and/or epilepsy is provided.

Screening Methods

The screening method of the present invention will generally involve screening candidate agents for their ability to open or activate a SK channel, particularly SK-1 which has been expressed by a cell (although mechanically based carriers are not excluded). The cell could naturally express the SK channel in which case further manipulation of the cell may not be needed. More preferably, however, a cell which does not normally express SK channels is transformed through recombinant DNA techniques to express (preferably overexpress) an SK channel. This maybe achieved according to techniques well known or apparent to those skilled in the art. Briefly, polynucleotides encoding the SK channel (which is preferably human but may be rat, murine or other mammalian species) are inserted into a suitable vector which in turn is inserted into the host cell by, for example, transfection. Suitable vectors maybe plasmid, phage, minichromosome or transposon of which plasmid is particularly preferred. The vector will typically include other regulatory elements such as a promoter, operator, activator, repressor and/or enhancer, transcription, translation, initiation and termination sequences. Particularly preferred are vectors which comprise a regulatory element or elements operably linked to the SK channel encoding polynucleotide to provide expression thereof at suitable levels for drug screening assays. Any of a wide variety of regulatory elements may be used and may be regulatable for example, being inducible. The term “operably linked” refers to a condition in which portions of a linear polynucleotide sequence are capable of influencing the activity of other portions of the same linear sequence, for example, a ribosome binding site is operably linked to a coding sequence if it is positioned so as to permit translation. Host cells expressing SK channels (whether naturally or through transformation) are typically eukaryotic and preferably mammalian such as HEK293 or CHO cells. It will of course be apparent to those skilled in the art that variants of the naturally occurring SK protein such as those wherein one or more amino acids has been substituted, deleted, inserted, rearranged or modified (particularly those involving what would be considered in the art as a conservative change) truncates and muteins are within the ambit of the present invention so long as they substantially retain the functional characteristics of the naturally occurring SK channel protein.

In broad terms, the screening method of the present invention involves contacting a functional SK channel, particularly SK-1, with a candidate agent and observing the response. This response can then be used to determine whether the candidate agent is a potential SK channel opener and such candidate agents may be selected and further developed for clinical use. It is preferred that the SK channel opener identified according to the present invention is selective for the SK channel family but more preferably selective for a SK channel sub-class such as SK-1. The SK channel expressing cell may be incubated in the presence of the candidate agent and the response measured by using conventional electrophysiological methods such as patch clamping or conventional spectroscopic methods such as a Fluorescence Image Plate Reader (Molecular Devices) FLIPR assay. Illustrative of commercial available plate based assays that may be employed in the screening method of the present invention include VIPR (Aurora Sciences), FLIPR (Molecular Devices) or the method involving bioluminescence resonance energy transfer (BRET) as disclosed in WO 99/66324 the entire contents of which are incorporated herein by reference and to which the reader is specifically referred.

Candidate agents may take the form of a small molecule organic chemical. The screening method maybe adapted for high throughput screening (HTS) according to methods well known or apparent to those skilled in the art. In this embodiment, a plurality of candidate agents is typically provided as a chemical library consisting of at least hundreds but usually many thousands of candidate agents. The library may comprise agents of high structural diversity or may comprise a constellation of structurally related agents. The constellation of agents may be used in a screen of the present invention (a so-called “focussed screen”) following an initial screen against a library of agents of high structural diversity which may provide information as to which constellation of agents should be investigated to find the most promising candidate agent(s). Of course the candidate agent(s) that are eventually selected from the screen (typically on the basis of ability to activate the target ion channel, e.g. Sk-1 and/or on selectivity profile for that target) maybe then be structurally modified in view of other parameters such as pharmacokinetics and/or toxicology profile before the agent is ready for administration into the patient in the form of a physiologically acceptable pharmaceutical composition as described infra.

Pharmaceutical Compositions

Pharmaceutical compositions comprising compounds of the invention (a term which encompasses compounds having the formula I, as well as agents identified in the screening methods described herein), also referred to herein as active ingredients, may be administered for therapy by any suitable route including oral, rectal, topical and parenteral (including subcutaneous, transdermal, intramuscular and intravenous). It will also be appreciated that the preferred route will vary with the conditions and age of the recipient and the chosen active ingredient.

The amount required of the individual active ingredient for the treatment of the disorder of course depends upon a number of factors including the severity of the symptoms of the disorder and the identity of the recipient and will ultimately be at the discretion of the attendant physician.

In general, for the foregoing conditions a suitable dose of a compound of the invention is in the range of from 0.05 to 100 mg per kilogram body weight of the recipient per day, preferably in the range of from 0.1 to 50 mg per kilogram body weight of the recipient per day, most preferably in the range of from 0.5 to 20 mg per kilogram body weight of the recipient per day and optimally from 1 to 10 mg per kilogram body weight per day. The desired dose is preferably presented as two, three, four, five, six or more sub-doses administered at appropriate intervals during the day. These sub-doses may be administered in unit dosage forms, for example, containing from 1 to 1500 mg, preferably from 5 to 1000 mg and most preferably from 10 to 700 mg of active ingredient per unit dosage form.

While it is possible for the active ingredient to be administered alone it is preferable to present it as a pharmaceutical composition. The compositions of the present invention comprise at least one active ingredient as defined above together with one or more acceptable carriers therefor and optionally other therapeutic agents. Each carrier must be “acceptable” in the sense of being compatible with the other ingredients of the composition and not injurious to the recipient. The test agents may be formulated with standard carriers and/or excipients as is routine in the pharmaceutical art, and as fully described in Remington's Pharmaceutical Sciences, Mack Publishing Company, Eastern Pennsylvania 17^{th} Ed. 1985.

Compositions include those suitable for oral, rectal, topical or parenteral (including subcutaneous, intramuscular, transdermal and intravenous) administration. The compositions may conveniently be presented in unit dosage form and may be prepared by any methods well known in the art of pharmacy. Such methods include the step of bringing into association the active ingredient with the carrier which constitutes one or more accessory ingredients. In general the compositions are prepared by uniformly and intimately bringing into association the active ingredient with liquid carriers or finely divided solid carriers or both, and then if necessary shaping the product.

Compositions of the present invention suitable for oral administration may be presented as discreet units such as capsules, cachets or tablets each containing a predetermined amount of the active ingredient; as powder or granules; as a solution or suspension in an aqueous or non-aqueous liquid; or as an oil-in-water liquid emulsion or in a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycollate, cross-linked povidone, cross-linked sodium carboxymethyl cellulose) surface-active or dispersing agent. Moulded tablets may be made by moulding in a suitable machine a mixture of powdered compound moistened with an inert liquid diluent. The tablets may be optionally coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for oral use as described above may also include buffering agents designed to neutralise stomach acidity. Such buffers may be chosen from a variety of organic or inorganic agents such as weak acids or bases admixed with their conjugated salts.

Compositions for rectal administration may be presented as a suppository with a suitable base comprising for example cocoa butter or a salicylate.

Compositions suitable for parenteral administration include aqueous and non-aqueous isotonic sterile injection solutions which may contain anti-oxidants, buffers, bacteriostats and solutes which render the composition isotonic with the blood of the intended recipient; and aqueous and non-aqueous sterile suspensions which may include suspending agents and thickening agents, as liposomes or other microparticulate systems which are designed to target the compounds to one or more organs. The compositions may be presented in unit-dose or multi-dose sealed containers, for example ampoules or vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Compositions suitable for transdermal administration may be presented as discreet patches adapted to remain in intimate contact with the epidermis of the recipient for a prolonged period of time. Such patches typically contain the active compound as an optionally buffered aqueous solution of, for example, from 0.1 to 0.2M concentration with respect to the said compound. As one particular possibility, the active compound, may be delivered from the patch by ionophoresis as generally described in Pharmaceutical Research 3(6), 318 (1986).

Preferred unit dosage compositions are those containing a daily dose or unit, daily sub-dose, as herein above recited, or an appropriate fraction thereof, of an active ingredient.

It should be understood that in addition to the ingredients particularly mentioned above the compositions of this invention may include other agents conventional in the art having regard to the type of composition in question, for example, those suitable for oral administration may include such further agents as sweeteners, thickeners and flavouring agents.

The compounds of the present invention can be prepared in any conventional manner, for example, as described in WO 95/30645, WO 95/30644 or WO 94/26693.

Use of a SK-1 ion channel activator other than a compound having the formula I, preferably identified by the screening method as described supra, in the manufacture of a medicament for the treatment of pain and/or irritable bowel syndrome and/or epilepsy is provided. Methods for the treatment of pain using said SK-1 ion channel activator is also provided.

The following Examples illustrate the invention.

### Example 1

Cloning of SK/IK Channels & Generation of Stable Cell Lines

The full length hSK-1, 2, 3 and IK clones were obtained either by PCR and 5′-RACE from a human cDNA library (Genbank accession numbers hSK1 U69883; hSK-2 unpublished) or from the Incyte or dbEST databases (Incyte Pharmaceuticals, Inc.; hSK3 AJ251016, hIK AF022797). An optimal Kozak sequence was added to the initiation site and the cDNA was cloned into Bluescript and subsequently the IRES vector pCIN5 for mammalian expression.

All SK/IK stable cell lines were generated in CHO—K1 using the pCIN5 construct, under selection for resistance to 500 μg/ml geneticin. Five μg of the purified plasmid DNA was used to transfect 5×10⁶ CHO—K1 cells (ATCC N° CRL 9618) by electroporation (standard protocol with BIORAD Gene Pulser II). After two days of growth in Petri dishes in alpha-MEM (Gibco BRL, Cat. No. 22571) and 10% FBS (Gibco BRL), chemical selection was initiated by addition of the antibiotic G418-sulfate (Calbiochem) at a final concentration of 0.5 mg/ml. After another two weeks of culture, 47 antibiotic-resistant clones were isolated, expanded under antibiotic selection and subsequently stored frozen in liquid nitrogen until further analysis.

Channel activity was determined either using electrophysiology (see later) or with a fluorescence assay using the membrane potential-sensitive fluorescence dye DiBAC₄(3) on a FLIPR machine (Molecular Devices). Cells were seeded in clear bottomed blacked walled sterile 96 well plates at 50,000 cells per well and grown at 37° C., 5% CO₂ for 2-3 days. Cells were then loaded with 5 μM DiBAC₄ for 45 min at 37° C. prior to addition of Ca²⁺ mobiliser (e.g. UTP) or ionomycin (1 μM) using the FLIPR pipettor head. A reduction in fluorescence is observed on opening the channel, equating to membrane hyperpolarisation. The cell line with the highest activity (eg greatest hyperpolarisation) for each channel was further dilution cloned, and again selected for channel activity. The presence of the correct channels was determined by RT-PCR using gene-specific primers and immunoblotting with specific antibodies. Stable cell lines were cultured in α-MEM (Gibco BRL, Cat.No 22571) with 10% heat inactivated foetal bovine serum (Gibco BRL, Cat.No 10108-165) at 37° C. and 10% CO₂.

### Example 2

Electrophysiology

Standard whole cell patch clamp electrophysiology methods are used to record membrane currents from single cells expressing either SK or IK channels. Cells are plated on to glass coverslips and placed in a small (volume <500 μl) chamber on the platform of an inverted microscope (Nikon Diaphot 200). A gravitational flow system is used to perfuse the cells (1 ml min⁻¹) with an external salt solution containing 144 mM KCl, 1 mM MgCl₂, 10 mM HEPES, 2 mM CaCl₂ buffered to pH 7.4 (osmolarity 300 mos). All experiments are performed at room temperature (20-22° C.). Membrane currents are amplified and recorded with an Axopatch 200B or HEKA EPC-9 patch clamp amplifier coupled to a Pentium microprocessor via an analog-digital interface. Patch pipettes are pulled (Sutter model 97) from 1.5 mm outside diameter borosilicate glass (Clark Electromedical) and fire polished (Narishige Microforge).to give final tip resistances of 2-4 mΩ. Pipettes are backfilled with an internal solution containing a 144 mM KCl, 10 mM EGTA, 10 mM HEPES, 1 mM MgCl₂ and concentrations of CaCl₂ calculated to give free Ca²⁺ concentrations ranging from 30 nM to 1 μM. A silver/silver chloride pellet is used as the bath (reference) electrode. Signals are pre-filtered at bandwidth 5 kHz and sampled at 20 kHz. Capacitance transients and series resistance errors are compensated for (80-85%) using the amplifier circuitry.

Voltage ramp protocols (−100 mV for 50 ms followed by a 1 s ramp to +100 mV) from a holding potential of 0 mV are applied to the cell to track the membrane current. Ramps are applied every 10 s. Non-specific ‘leak current’ is defined by the residual current at 0 mV when the external solution is replaced by one containing zero K+ (144 mM NaCl, 1 mM MgCl₂, 10 mM HEPES, 2 mM CaCl₂ buffered to pH 7.4 (osmolarity 300 mos)). Once a stable, baseline Ca²⁺-activated K⁺ current is recorded (usually 50-500 pA in amplitude, 3-5 min after whole cell recording configuration is achieved) drug solutions are applied to the cell via a ‘U-tube’ system. This is comprised of a fine microfil tube positioned within 100 μM of the cell, attached to two solenoid valves and a reservoir of drug containing solution.

Application of drugs typified by a, b, c, and d at concentrations of 10 μM increased the whole cell K⁺ current in SK/IK cells but not in untransfected cells, indicating that they are specific SK/IK openers. The effects of these agents are concentration-related, typically between 100 nM-300 μM. Generally the effects could be reversed on washout.

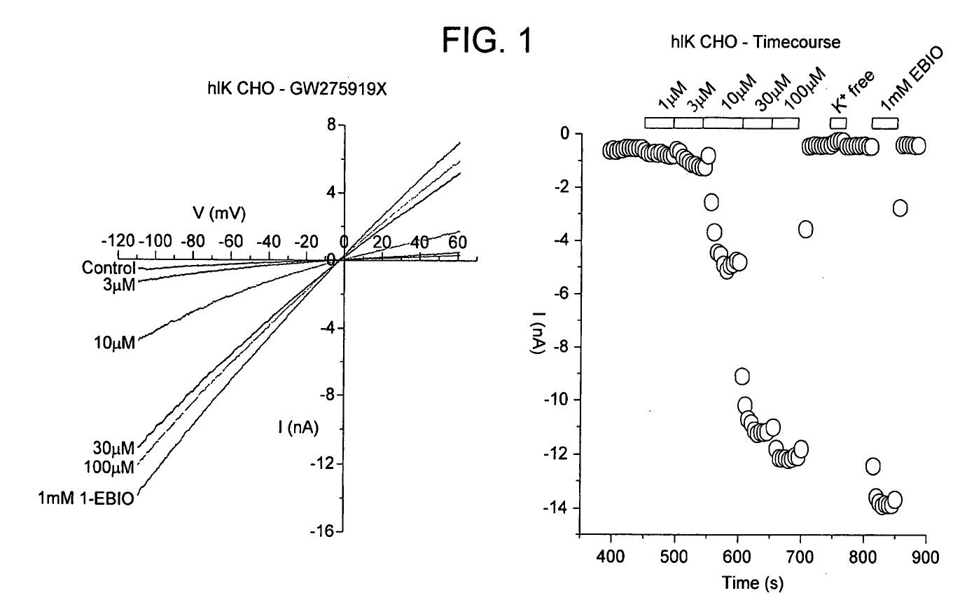

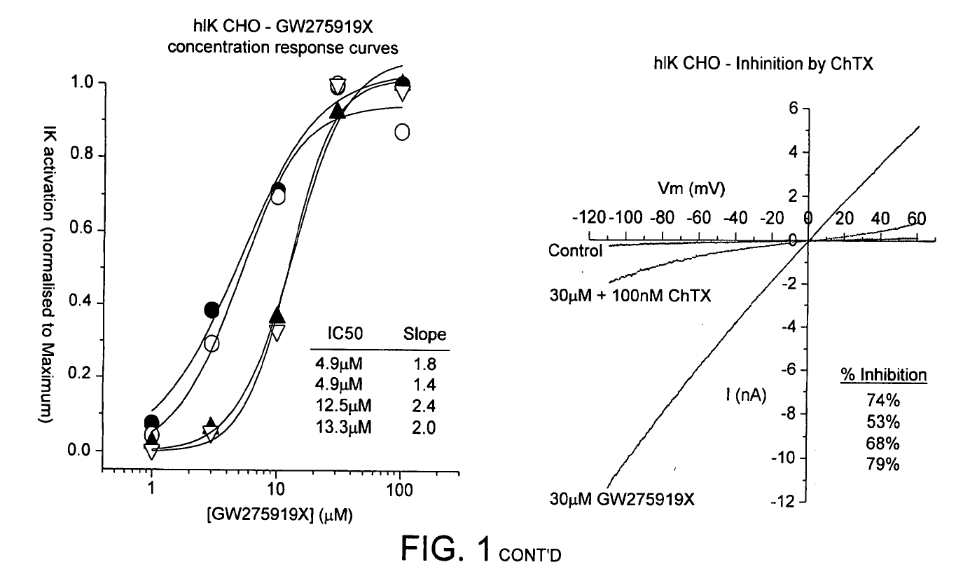

In some experiments the effect of internal Ca²⁺ concentrations on the potency of drugs typified by a,b,c and d was explored by comparing responses between cells recorded with either 30 nM, 100 nM or 300 nM free Ca²⁺ in the pipette solution. Typically, lower concentrations of openers were required to activate channels when Ca2+ concentrations were high i.e. the activity of these agents may be Ca²⁺-sensitive. A representative data set is shown in FIG. 1.

### Example 3

Screening Using Voltage-Sensitive Dyes

To screen for novel SK-1 openers FLIP-R DiBAC experiments are conducted in either 96-well or 384-well format. hSK-1 CHO (or HEK) cells were seeded in clear bottomed blacked walled sterile well plates at 50,000 cells per well (96) and grown at 37° C., 5% CO² for 2-3 days. Cells were then loaded with 5 μM DiBAC₄ for 45 mins at 37° C. and then read on FLIPR machine (Molecular Devices) for fluorescent intensity. Test compounds were added on line, usually in volumes of 10-50 μl using the FLIPR pipettor head (final test concentrations usually from 1 pM-30 pM). The DiBAC fluorescence is monitored for a further 10 mins. Openers of SK-1 channels recduce fluorescence in a concentration-dependent manner, which is interpreted as membrane hyperpolarisation. In wild-type untransfected cells ‘true’ openers of SK-1 channels (as opposed to dye ‘quenchers’ or non-specific membrane hyperpolarising agents) do not change DIBAC fluorescence. Selectivity for the SK-1 channel (preferably>10-fold) is assessed by conducting similar experiments in stable cell lines that have been transfected with either hSK-1, hSK-3 or hMK Ca²⁺ activated K+ channels.

### Example 4

Screening Using Electrophysiology

Standard whole cell patch clamp electrophysiology methods are used to record membrane currents from single cells expressing SK-1 channels. Cells are plated on to glass coverslips and placed in a small (volume<500 μl) chamber on the platform of an inverted microscope (Nikon Diaphot 200). A gravitational flow system is used to perfuse the cells (1 ml min⁻¹) with an external salt solution containing 144 mM KCl, 1 mM MgCl₂, 10 nmM HEPES, 2 mM CaCl₂ buffered to pH 7.4 (osmolarity 300 mos). All experiments are performed at room temperature (20-22° C.). Membrane currents are amplified and recorded with an Axopatch 200B of HEKA EPC-9 patch clamp amplifier coupled to a Pentium microprocessor via an analog-digital interface. Patch pipettes are pulled (Sutter model 97) from 1.5 mm outside diameter borosilicate glass (Clark Electromedical) and fire polished (Narishige Microforge) to give final tip resistances of 2-4 mΩ. Pipettes are backfilled with an internal solution containing a 144 mM KCl, 10 mM EGTA, 10 mM HEPES, 1 mM MgCl₂ and concentrations of CaCl₂ calculated to give free Ca²⁺ concentrations ranging from 30 mM to 1 μM. A silver/silver chloride pellet is used as the bath (reference) electrode. Signals are pre-filtered at bandwidth 5 kHz and sampled at 20 kHz. Capacitance transients and series resistance errors are compensated for (80-85%) using the amplifier circuitry.

Voltage ramp protocols (−100 mV for 50 ms followed by a 1 s ramp to +100 mV) from a holding potential of 0 mV are applied to the cell to track the membrane current. Ramps are applied every 10 s. Non-specific ‘leak current’ is defined by the residual current at 0 mV when the external solution is replaced by one containing zero K+ (144 mM NaCl, 2 mM MgCl₂, 10 mM HEPES, 2 mM CaCl₂ buffered to pH 7.4 (osmolarity 300 mos). Once a stable, baseline Ca²⁺ -activated K+ current is recorded (usually 50-500 pA in amplitude, 3-5 min after whole cell recording configuration is achieved) drug solutions are applied to the cell via a ‘U-tube’ system. This is comprised of a fine microfil tube positioned within 100 μM of the cell, attached to two solenoid valves and a reservoir of drug containing solution. SK-1 channels openers are detected by application to the cell for periods of between 5 s and 10 mins at concentrations usually ranging from 1 nM-100 μM. Openers increase the whole cell K+ current in SK/IK cells but not in untransfected cells, indicating that they are specific. The effects of these agents are concentration-related, typically between 100 nM-300 μM. Generally the effects can be reversed on washout.

### Example 5

Drug Screening in Animal Models

Efficacy of SK-1 openers in animal models of neuropathic and/or inflammatory pain can be determined using standard techniques e.g. the chronic constriction injury (CCI) model (Bennett & Xie, 1988 Pain, 33, 87-107) or intraplantar administration of carrageenan or Complete Freunds adjuvant (CFA). For example, in CCI, the common left sciatic nerve of male Random Hooded rats (180-200 g) was exposed at mid thigh level under Isoflurane anaesthesia. Four ligatures of chromic gut (4.0) were tied loosley around the nerve with a 1 mm spacing between each. The wound was then closed and secured with suture clips. The surgical procedure was identical for the sham operated animals except the sciatic nerbe was not ligated. The animals were allowed a period of 12 days to recover from the surgery before any behavioural testing began. The effect of test compounds on CCI-induced decrease in mechanical paw withdrawal threshold was measured using an algesymeter (Randall & Selitto, 1957). The prescence of mechanical allodynia was assessed using Von Frey Hair monofilaments (range: 4.19-84.96 g). The rats were lightly restrained and placed upon a metal grid floor, from below which the monofilaments were applied to the plantar surface of the hindpaws. The lowest monofilament to produce a withdrawal was the response recorded. The effect of drugs on allodynia and/or mechanical paw withdrawal threshold was assessed by dosing the animals either acutely or chronically preferably via either the p.o., i.p., or s.c. route.

Intraplantar administration of Freund's Complete adjuvant (FCA; Mycobacterium tuberculosis [1 mg/ml] 100 μl) in rats produces a reduction in weight bearing (a measure of incident pain/hyperalgesia) on the injected rat paw and an associated oedema (acute inflammatory response). The effects of SK-1 openers are investigated in the ‘actue’ monoarthritis protocol (drugs administered one hour prior to FCA; hyperalgesia and oedema assessed 6 hours post FCA) and 2 days post-inflammatory insult (‘established’ monoarthritis). Carrageenan, when dosed intraplantar in the rat, induces a characteristic injury and inflammation of the paw, leading to spontaneous pain, (allodynia) and inflammation (oedema), which peak at about 3 hrs. Fasted rats are dosed with either vehicle or test compound (preferably p.o., s.c. or i.p.) and thirty minutes later are briefly anaesthetised, with isoflurane. 0.0 ml of 2% carrageenan solution is injected into the sub plantar surface of the left hind paw. Rats are observed until recovery from anaesthetic. Three hours after injection of carrageenan the rats are placed individually into the clear Perspex restraint on the weight bearing machine. It is checked that the rat is standing with his hind paws correctly located on the 2 individual weight bearing platforms. The average weight distribution between the two platforms for a period of 8 seconds is assessed.

Efficacy of SK-1 openers in visceral pain models can be evaluated using a pseudoeffective cardiovascular reflex response to colonic distension. Fasted AHA rats of either sex (280 -340 g) were anaesthetised with pentobarbitone (60 mgkg⁻¹ i.p.). The femoral veins and a femoral artery were cannulated for anaesthetic administration, drug administration and blood pressure measurements, respectively. The trachea was also cannulated. The colon was washed out with 10 mls saline before insertion of a 6 cm balloon, 1 cm from the anus. Anaesthesia was maintained with a 20 mgkg⁻¹ hr continuous infusion of pentobarbitone. 80 mmHg phasic distensions were performed every 5 mins for 20 seconds, each of which produces a reflex drop in blood pressure. SK-1 openers are administered I.V. between distensions, 2½ minutes after the previous distension. Four consecutive, reproducible control responses were obtained before drugs were administered except in antagonist studies. In this case, once stable responses were obtained antagonist was administered (I.V.) 15 mins prior to agonist (I.V.). SK-1 openers should reduce the distension-evoked drop in blood pressure.

1. A compound of formula (I): 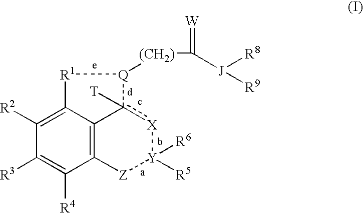
wherein
R², R³ and R⁴, which are the same or different are each hydrogen, hydroxy, halogen, cyano, C₁₋₆ alkyl, C₁₋₆ alkoxy or C₁₋₆ haloalkyl;
R⁸ and R⁹, which are the same or different are each hydrogen, C₃₋₅ cycloalkyl, C₁₋₆ alkoxy, phenyl, benzyl or C₁₋₆ alkyl which is unsubstituted or substituted by hydroxy; or R⁸ and R⁹ together with \dot{J} form a morpholino group, a pyrrolidine group or an azetidine group;
n is 0 or 1;
W is O or S; and
J is O or N; and
(a) _{——}e_{——} is a single bond, _{——}d_{——} is a double bond, and _{——}a_{——}, _{——}b_{——} and _{——}c_{——} are absent;
R⁵, R⁶, T and Y are absent;
R¹ is CH₂ or O;
X is hydrogen or C₁₋₆ alkyl; Z is as defined above for R², R³ and R⁴; and
Q is carbon, or
(b) _{——}e_{——} is absent;
_{——}d_{——} is a single bond or a double bond, _{——}c_{——} is a bond or is absent,
provided that when _{——}d_{——} is a double bond, _{——}c_{——} and T are absent; and
when _{——}d_{——} is a single bond one of T and _{——}c_{——} is absent;
_{——}b_{——} is a single bond and _{——}a_{——} is a single bond or is absent, or
_{——}a_{——} Y(R⁵)(R⁶)_{——}b_{——} represents a single bond;
Q is CH when _{——}d_{——} is a double bond and CH₂ when _{——}d_{——} is a single bond;
T is hydrogen or hydroxy;
X is CR⁷ or CHR⁷, wherein R⁷ is hydrogen, hydroxy, halogen, nitroxy, C₁₋₆ acyloxy or C₁₋₄ alkoxy;
Y is C or CH;
Z is (i) CH₂, oxygen or sulphur when _{——}a_{——} is a bond; or (ii) is hydrogen, halogen or C₁₋₆ alkyl when _{——}a_{——} is absent; or (iii) the moiety Z_{——}a_{——} represents a bond when R⁵ and R⁶ together with Y form a carbonyl group;
R¹ is as defined above for R², R³ and R⁴;
R⁵ is hydrogen or C₁₋₆ alkyl;
R⁶ is hydrogen, C₁₋₆ alkyl, hydroxy or halogen, or R⁵ and R⁶ together with Y form a carbonyl group; or
(c) _{——}e_{——} is absent; _{——}d_{——} is a single bond or a double bond, provided that when _{——}d_{——} is a double bond, T is absent;
_{——}c_{——} is absent; and
_{——}a_{——} Y(R⁵)(R⁶)_{——}b_{——} is absent;
Q and T are as defined in (b) above;
X is hydrogen or C₁₋₆ alkyl;
Z is hydrogen, halogen or C₁₋₄ alkyl; and
R¹ is as defined above for R², R³ and R⁴;
or a salt, solvate or physiologically functional derivative thereof for use as a IK and/or SK channel opener.

2. Use of a compound according to claim 1 in the manufacture of a medicament for the treatment of a disorder responsive to enhanced IK and/or SK channel activity.

3. Use according to claim 2 wherein the disorder is selected from a urinogenital disorder, a respiritory disorder, a cardiovascular disorder, a neuronal hyperexcitability disorder, sickle cell anaemia, sleeping disorders, a disorder of the central nervous system, inflammation, pain and inflammatory bowel disease (IBD).

4. Use according claim 2 or 3 wherein the urinogenital disorder is selected from bladder hyperexcitability, MED and urinary incontinence.

5. Use according claim 2 or 3 wherein the respiritory disorder is selected from asthma, chronic obstructive pulmonary disease (COPD) and cystic fibrosis.

6. Use according claim 2 or 3 wherein the cardiovascular disorder is selected from hypertension, angina pectoris, ischaemic heart disease and cerebral ischaemia.

7. Use according to claim 2 or 3 wherein the neuronal hyperexcitability disorder is selected from irritable bowel syndrome, bipolar disorder and psychosis.

8. A method for treating a patient afflicted with a disorder responsive to enhanced IK and/or SK channel activity, the said method comprising administering to a patient in need of treatment a therapeutically effective amount of a compound of formula (I): 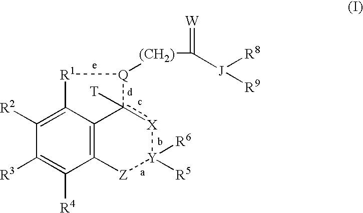
wherein
R², R³ and R⁴, which are the same or different are each hydrogen, hydroxy, halogen, cyano, C₁₋₆ alkyl, C₁₋₄ alkoxy or C₁₋₆ haloalkyl;
R⁸ and R⁹, which are the same or different are each hydrogen, C₃₋₅ cycloalkyl, C₁₋₆ alkoxy, phenyl, benzyl or C₁₋₆ alkyl which is unsubstituted or substituted by hydroxy; or R⁸ and R⁹ together with J form a morpholino group, a pyrrolidine group or an azetidine group;
n is 0 or 1;
W is O or S; and
J is O or N; and
(a) _{——}e_{——} is a single bond, _{——}d_{——} is a double bond, and _{——}a_{——}, _{——}b_{——} and _{——}c_{——} are absent;
R⁵, R⁶, T and Y are absent;
R¹ is CH₂ or O;
X is hydrogen or C₁₋₆ alkyl; Z is as defined above for R², R³ and R⁴; and
Q is carbon, or
(b) _{——}e_{——} is absent;
_{——}d_{——} is a single bond or a double bond, _{——}c_{———} is a bond or is absent,
provided that when _{——}d_{——} is a double bond, _{——}c_{——} and T are absent; and
when _{——}d_{——} is a single bond one of T and _{——}c_{——} is absent;
_{——}b_{——} is a single bond and _{——}a_{——} is a single bond or is absent, or
_{——}a_{——} y(R⁵)(R⁶)_{——}b_{——} represents a single bond;
Q is CH when _{——}d_{——} is a double bond and CH₂ when _{——}d_{——} is a single bond;
T is hydrogen or hydroxy;
X is CR⁷ or CHR⁷, wherein R⁷ is hydrogen, hydroxy, halogen, nitroxy, C₁₋₆ acyloxy or C₁₋₄ alkoxy;
Y is C or CH;
Z is (i) CH₂, oxygen or sulphur when _{——}a_{——} is a bond; or (ii) is hydrogen, halogen or C₁₋₆ alkyl when _{——}a_{——} is absent; or (iii) the moiety Z_{——}a_{——} represents a bond when R⁵ and R⁶ together with Y form a carbonyl group;
R¹ is as defined above for R², R³ and R⁴;
R⁵ is hydrogen or C₁₋₆ alkyl;
R⁶ is hydrogen, C₁₋₆ alkyl, hydroxy or halogen, or R⁵ and R⁶ together with Y form a carbonyl group; or
(c) _{——}e_{——} is absent; _{——}d_{——} is a single bond or a double bond, provided that when _{——}d_{——} is a double bond, T is absent;
_{——}c_{——} is absent; and
_{——}a_{——} Y(R⁵)(R⁶)_{——}b_{——} is absent;
Q and T are as defined in (b) above;
X is hydrogen or C₁₋₆ alkyl;
Z is hydrogen, halogen or C₁₋₆ alkyl; and
R¹ is as defined above for R², R³ and R⁴;
or a salt, solvate or physiologically functional derivative thereof.

9. A method according to claim 8 wherein the disorder is selected from a urinogenital disorder, a respiritory disorder, a cardiovascular disorder, a neuronal excitability disorder, sickle cell anaemia, sleeping disorders, hyperalkaemia, a disorder of the central nervous system, inflammation, pain and inflammatory bowel disease (IBD).

10. Use according claim 8 or 9 wherein the urinogenital disorder is selected from bladder hyperexcitability, MED and urinary incontinence.

11. Use according claim 8 or 9 wherein the respiritory disorder is selected from asthma, chronic obstructive pulmonary disease (COPD) and cystic fibrosis.

12. Use according claim 8 or 9 wherein the cardiovascular disorder is selected from hypertension, angina pectoris, ischaemic heart disease and cerebral ischaemia.

13. Use according claim 8 or 9 wherein the neuronal hyperexcitability disorder is selected from irritable bowel syndrome, bipolar disorder and psychosis.

14. A method for the identification of a candidate agent useful in, inter alia, the treatment of a disease or disorder selected from the group consisting of; pain, urinogenital disorders such as bladder hyperexcitability, MED and urinary incontinence, respiratory disorders such as asthma, chronic obstructive pulmonary disease (COPD) and cystic fibrosis, cardiovascular disorders such as hypertension, angina pectoris, ischaemic heart disease and cerebral ischaemia, neuronal hyperexcitability disorders such as irritable bowel syndrome, bipolar disorder and psychosis, sickle cell anaemia, sleeping disorders, hyperalkaemia inflammation, pain and inflammatory bowel disease (IBD) which method comprises the steps of;
(a) providing a candidate agent;
(b) contacting a SK-1 channel with said candidate agent;
(c) selecting a candidate agent which modulates opening of said SK-1 channel;
(f) optionally synthesising and/or purifying said agent of step (c).

15. A method for the treatment of a disease or disorder selected from the group consisting of; pain, urinogenital disorders such as bladder hyperexcitability, MED and urinary incontinence, respiratory disorders such as asthma, chronic obstructive pulmonary disease (COPD) and cystic fibrosis, cardiovascular disorders such as hypertension, angina pectoris, ischaemic heart disease and cerebral ischaemia, neuronal hyperexcitability disorders such as irritable bowel syndrome, bipolar disorder and psychosis, sickle cell anaemia, sleeping disorders, hyperalkaemia inflammation, pain and inflammatory bowel disease (IBD) which method comprises the steps of;
(a) providing a candidate agent;
(b) contacting a SK-1 channel with said candidate agent;
(c) selecting a candidate agent which modulates (i.e. promotes) opening of said SK-1 channel;
(d) optionally synthesising and/or purifyng said agent of step (c);
(e) administering said agent of step (c) or (d) to a mammalian patient, particularly a human, in clinical need thereof.

16. The method of claim 14 or 15 wherein the disease or disorder is pain.
